(19) United States
(12) Patent Application Publication
(10) Pub. No.: US 2005/0234248 A1
(43) Pub. Date: Oct. 20, 2005

(54) Method of producing preparations rich in tocotrienol

(75) Inventors: Peter Kossler, Mariapfarr (AT); Norbert Fuchs, Mariapfarr (AT); Behzad Sadeghi, Vienna (AT)

Correspondence Address: FULBRIGHT & JAWORSKI L.L.P., 600 CONGRESS AVE., SUITE 2400, AUSTIN, TX, 78701, US

(73) Assignee: Vis-Vitalis Lizenz- Und Handels Gmbh

(21) Appl. No.: 10/513,319

(22) PCT Filed: Apr. 30, 2003
(86) PCT No.: PCT/AT03/00125

§ 371 Date: May 20, 2005

(30) Foreign Application Priority Data
May 3, 2002 (AT) A6852002

(51) Int. Cl. C07D 311/74; C07D 311/76

(52) U.S. Cl. 549/413

(57) ABSTRACT

The invention relates to a method for producing preparations rich in tocotrienol, comprising the following steps: incubation of plant seeds with an electrolytic nutrient solution to form plant seedlings rich in tocotrienol; harvesting of plant seedlings and extraction of a preparation containing tocotrienol from the harvested plant seedlings, in particular from the bran and the germ of the harvested plant seedlings.

The invention relates to a method of producing tocotrienol-enriched preparations.

While over the last decades, conventional nutritional science has primarily been confined to the assaying of nutrients (proteins, carbohydrates, fats, vitamins, mineral substances and micronutrients) for their “nutritive” properties only (i.e. for ensuring the constructional and operational metabolisms), in recent years the interest of nutritional science has increasingly focussed on so-called secondary plant substances (phytochemicals). Although these natural substances by definition are not counted among the classical nutrients, they still have numerous biological effects, and therefore they are also termed “bioactive plant substances”.

Tocotrienols belong to this category of plant substances, even though they are also counted among the vitamins because they exhibit vitamin E activity, even though a slight one. Apart from this vitamin E activity, tocotrienols have cholesterol-lowering, cell-protective and antioxidant properties. Antioxidants are characterised by their ability to transfer electrons to partner molecules on molecular level, and this ability to transfer electrons for defined individual molecule compounds is quantified by the so-called standard redox potential, yet for antioxidant mixtures it is expressed by the so-called antioxidant capacity (reductive capacity). Compared to synthetic tocopherols, the antioxidant potential of tocotrienols—depending on the assaying medium and the assaying method—is described to be fifty to one-thousand times higher.

Due to their molecular structure, tocotrienols are exclusively fat-soluble, unfolding their biological activity in human and animal tissues primarily at the lipophilic compartments (intra- and extracellular biomembranes). Lipophilic antioxidants therefore play an important biological role within the scope of the antioxidant protection of nuclei (genetic material), the mitochondria (cellular energy supply), the endoplasmatic reticulum (cellular synthesis performance) as well as on the cell membrane (stability and lifetime of tissues). For this reason, the tocotrienols are of an enormous importance, beyond their basic nutritional purpose, for the protection of the genetic material (protection against mutations by damaging peroxides, radicals and xenobiotics), for the optimum cellular energy supply (capability of immune and organ cells), for the cellular synthesis performance (regenerating potential of the immune system and of tissues) as well as for the functioning ability and lifetime of all body cells. In particular, also the nervous system, the central nervous system just as the peripheral nervous system which consists by more than 50% of lipoid substances, relies on a sufficient and permanent protection of its structures by lipophilic antioxidants. The nutrition-medical fields of application of tocotrienols therefore include the immune system (allergies, cancer) just as the cardiovascular system (Angina pectoris, cardiac infarction prevention and aftercare), the muscle/tendon/joint system (degenerative myopathies and arthropathies), the liver as detoxicating organ (environmentally-caused diseases), the skin (atopic diseases, ageing), and finally, degenerative processes of the nervous system (Multiple Sclerosis, Morbus Alzheimer, Morbus Parkinson, spinal and peripheral-neurological diseases and trauma sequelae).

Therefore, there is a demand for providing tocotrienol-containing products, in particular natural products having particularly high tocotrienol contents.

Thus, the invention relates to a method of producing tocotrienol-enriched preparations, said method comprising the following steps:
incubating plant seeds with an electrolyte nutrient solution so as to form tocotrienol-enriched plant embryos,
recovering the plant embryos, and
extracting a tocotrienol-containing preparation from the recovered plant embryos, in particular from the bran and from the germs of the recovered plant embryos.

It has been shown that the tocotrienol content of plant embryos can be decisively increased with the method according to the invention, even though other, closely related antioxidant secondary plant substances, such as, e.g., tocopherols, in part are greatly reduced during germination, particularly during the germination effected according to the invention. In principle, it is generally assumed in the course of germination that the germinating seed's demand of building material and antioxidant protection molecules for this process increases, yet that this increased demand is immediately covered either by the substances present or by newly formed substances. It was the more surprising that a certain, particularly valuable class of compounds, the tocotrienols, could be selectively enriched by germination in an electrolyte-enriched nutrient solution.

The germination of plant seeds in electrolyte nutrient solutions has been described in EP 0 770 324 A and EP 0 799 578 A, e.g. It has been-known that the plant embryos obtained there do exhibit an increased electrolyte content, but that the consumption of antioxidant protective molecules is also increased and their content in the plant embryo therefore is reduced.

According to U.S. Pat. No. 5,908,940 A, a special method of preparing “Tocol”-products (tocotrienol, tocopherol and tocotrienol-like compounds) is described in which plant raw material is dry heated for 30 min to 4 h at 80-150° C., whereupon the desired ingredients are extracted. As the plant starting material, a number of the most varying materials is mentioned. Yet, neither germination of the starting material (for preparing the electrolyte-enriched plant embryos) nor the enrichment of the starting material with inorganic nutrient substances is provided for. Much rather, the method described there aims at inactivating plant enzymes by the action of heat so as to prevent a degradation of tocopherols and tocotrienols. The central, surprising aspect of the present invention according to which precisely tocotrienols can be recovered in an increased yield in special electrolyte-enriched plant embryos with, however, the tocopherol content, e.g., being lowered at the same time, neither appears from this document nor is it rendered obvious by this document.

EP 0 616 810 A1 relates to the use of germinating rice as a medicine, in particular for the prophylaxis and therapy of cancer. In this document, however, neither a general reference is made to a tocotrienol content, nor can any hint be read therefrom that precisely in (rice) embryos an increased tocotrienol content can be found. Moreover, the germination in electrolyte-enriched media does not appear from this document.

The electrolyte nutrient solution preferably contains—independently of each other—1 mg/l or more, preferably 10 mg/l or more, in particular 50 mg/l or more, of zinc, iron, potassium and/or magnesium ions, 0.5 mg/l or more, preferably 5 mg/l or more, in particular 25 mg/l or more, of copper, manganese, strontium and/or lithium ions, 0.1 mg/l or more, preferably 1 mg/l or more, in particular 5 mg/l or more, of selenium, molybdenum, chromium, arsenic, vanadium and/or cobalt ions.

In principle, the method according to the invention is applicable to many different types of plant seeds, according to the invention, however, naturally seeds are preferred which either enable particularly high tocotrienol contents, or seeds from plants which are particularly well suited for a large industrial realisation of the method according to the invention. Therefore, preferably, the plant seeds are selected from walnut, wheat, sunflower, palm, rye, barley, oat, amaranth, quinoa, rice seeds or mixtures of these seeds.

Preferably, the plant embryos are dried prior to extraction. This not only increases their storability, but also makes the plant embryos obtained according to the invention suitable for many large-scale applications.

Moreover, it is preferred if the plant embryos are ground prior to extraction, since the content of tocotrienols determining their value, and of other lipophilic antidoxidants as well as essential fatty acids in the bran and in the germs, is the highest. In this manner, the plant embryos can better be transferred to and extracted in well-established (oil) extraction plants, in particular of the type having pressure separators.

Due to their chemical structure, tocotrienol preparations preferably are recovered as an oil. Extraction, therefore, preferably is performed by obtaining an oily extract. Although an extraction by means of organic solvents (even with organic solvents that are food-technologically harmless) or in (aqueous) suspensions (e.g. with micelles etc.) is possible, it is not preferred according to the invention, since the inventive tocotrienol preparations preferably are to be provided as natural, unfalsified and biologically valuable as possible. For this, it is also suitable to co-extract as much as possible of the natural reaction partners of the tocotrienols so as to obtain as high a biological effect as possible during the application on humans.

Therefore, particularly preferably the extraction according to the invention is effected with supercritical CO₂. The fat-soluble components may, however, also be extracted with hexane or other organic solvents.

Since the tocotrienol content of the plant embryos may decrease again if incubation with the nutrient solution lasts too long, it is necessary that the duration of incubation with the nutrient solution is optimised for each type of seed. This, however, will be easily possible for any person skilled in the art, e.g. by the incubation and analysis methods disclosed in the following example section. Preferably, the duration of incubation will be chosen such that an optimum content of tocotrienols can be obtained. Preferably, it will be incubated until the content of tocotrienols will be increased by at least 100%, in particular by at least 300%, relative to their content in non-germinated seeds.

Incubation will be effected at temperatures and under conditions that are suitable or have proved successful for the conventional germination of seeds of the selected type. According to a preferred embodiment, incubation according to the invention is carried out at a temperature of from 10 to 40° C., preferably from 15 to 30° C., in particular 19 to 21° C.

The electrolyte nutrient solution with which the plant seeds are incubated preferably contains vanadium, selenate, molybdate, cobalt, chromium(III), manganese, strontium, lithium, copper iron(III), zinc, gluconate, citrate, lactate ions, or combinations of these ions, in an amount of from 0.1 to 1000 mg, preferably from 1 to 500 mg, in particular from 3 to 100 mg.

The extraction according to the present invention may be achieved with a plurality of suitable devices and methods, each adapted to the chosen seeds or plant embryos, respectively. According to the invention, extraction by means of autoclaves and pressure separators has proved particularly suitable. The latter may preferably, independently, be operated at an autoclave pressure of 100 bar or more, preferably 200 bar or more, in particular 250 bar or more, at a separator pressure of 20 bar or more, preferably 30 bar or more, in particular 45 bar or more, at an autoclave temperature of 30° C. or more, preferably 40° C. or more, in particular 50° C. or more, and at a separator temperature of 20° C. or more, preferably 30° C. or more, in particular 40° C. or more.

Depending on the duration of incubation and consumption of the nutrient solution, incubation in preferred instances may be carried out by at least once, preferably at least twice, in particular at least three times changing the nutrient solution.

In a further aspect, the present invention relates to tocotrienol-enriched plant embryos or tocotrienol-enriched preparations, obtainable by the method according to the invention. Particularly preferred are tocotrienol-enriched wheat embryos or tocotrienol-enriched wheat embryo preparations having a tocotrienol content of 500 mg/kg dry material or more, preferably of 1000 mg/kg dry material or more, in particular of 2000 mg/kg dry material. Moreover, also tocotrienol-enriched barley embryos or tocotrienol-enriched barley embryo preparations are preferred which exhibit a tocotrienol content of 1500 mg/kg dry material or more, preferably a gamma-tocotrienol content of 500 mg/kg dry material, in particular a gamma-tocotrienol content of 200 mg/kg dry material.

From the inventive tocotrienol-enriched plant embryos or the tocotrienol-enriched preparations, tocotrienol-containing preparations can be prepared which address a nutritionally-scientifically particularly important aspect, and which are capable of acting particularly as a biologically valuable effective antioxidant since in them the increased tocotrienol content not only acts as an isolated (increased) tocotrienol dose, but also by the fact that the tocotrienols which, according to the invention, can be administered with their natural partners of action (in particular, redox-partners), with these partners are also much more effective in their action.

Depending on the respective demand, the tocotrienol-containing preparation according to the present invention may preferably additionally be admixed with pharmaceutically active substances, pharmaceutical adjuvants, food-technological products or food-technological additives. In doing so, however, preferably care should be taken that with such an addition the “natural balance” between tocotrienols and the natural partners of action mentioned is not substantially negatively affected.

The present invention will be explained in more detail by the following examples to which, however, it shall not be restricted.

### EXAMPLE 1

Method of Recovering Tocotrienol-Rich Oils From Germinating Cereals:

Certain plant oils, such as walnut oil, wheat germ oil or sunflower oil, are considered to be particularly rich in tocopherols, in particular D,L-beta-tocopherol. High contents of antioxidant tocotrienols, however, are found in palm oil as well as in rye, barley, oat, wheat bran and rice. Germinating cereal and leguminose seeds are known to increase their vitamin contents in an endogenous manner which accounts for the increased synthesis performance during the germination process.

In addition, in the course of the increased new cell formation, also the polyunsaturated fatty acids increase by nearly 50% so as to provide sufficient biologically valuable “building material” for new cell formation. Polyunsaturated fatty acids are, however, highly oxidation-sensitive relative to light and oxygen so that germinating seeds synthesize also appropriate amounts of lipophilic antioxidants (tocotrienols) for the protection of these biologically valuable fatty acids. The present tests aimed at determining the changes of tocopherol and tocotrienol contents during the germination process in terms of quality and quantity and at searching for possible ways of purposefully increasing particularly the contents of antioxidant tocotrienols during the germination process.

The following Table 1 shows the gas chromatographically determined contents of (highly) unsaturated fatty acids of the “barley bran oil, germinated for 24 hours with nutrient solution” indicated-in Table 5.

TABLE 1
Chemical Tests

| Type | Values |
|---|---|
| Water (grav.) % | 4.9% |
| Acid number (titr.) | 8.8 |
| Free fatty acid calc. % | 4.4% |
| Peroxide number according to Wheeler | 4.1 |

Composition of the fatty acid methyl ester mixture:
(gas chromatographically)

| | |
|---|---|
| Palmitic acid | 19.8% |
| Stearic acid | 1.1% |
| Oleic acid | 19.5% |
| Linoleic acid | 53.6% |
| Linolenic acid | 4.1% |
| Arachic acid | 0.3% |
| Behenic acid | 0.3% |
| Erucic acid | 0.1% |
| Lignoceric acid | 0.2% |
| Icosapentenoic acid (C20 5=) | 0.2% |
| Ash (grav.) | <0.1 g/100 g |
| Fat (acc. to Weibull) | 94.6 g/100 g |
| Saturated fatty acids | 20.5 g/100 g |
| Monounsaturated fatty acids | 19.3 g/100 g |
| Polyunsaturated fatty acids | 54.8 g/100 g |
| Petroleum ether insoluble (material) | 1.0% |
| Soap content | 0.3% |

Germination Tests:

Germinative wheat and barley seeds were alternatively germinated with distilled water or with a nutrient solution for a period of time of 24 or 96 hours, respectively.

The nutrient solution contained the following dissolved nutrient salts (data in mg/l):

TABLE 2

| | |
|---|---|
| Vanadium oxide sulfate 5 H₂O | 24.85 |
| Sodium selenate | 11.95 |
| Sodium molybdate | 12.60 |
| Cobalt chloride 6 H₂O | 20.20 |
| Chromium-III-chloride | 25.60 |
| Manganese chloride | 73.75 |
| Strontium lactate | 84.25 |
| Lithium chloride | 152.75 |
| Copper gluconate | 178.50 |
| Ammonium-iron III-citrate | 178.50 |
| Zinc gluconate | 394 |

Prior to the germination phase proper, the cereal seeds were soaked in the respective solutions for twelve hours. Germination was effected at room temperature (19-21° C.) and under normal day/night light conditions in commercial germinators which consisted of transparent, superposed plastic dishes with draining means. During germination, the plant embryos were rinsed twice per day with the respective solutions (i.e., distilled water or nutrient solution, respectively, at 250 ml/90 g each). After their harvest, the plant embryos were thoroughly rinsed with twice distilled water (three times, with approximately 800 ml), and subsequently dried at 60° C. under hot air. After the drying process, the germinated seeds were ground, and the brans obtained therefrom were extracted by means of supercritical CO₂.

The extraction parameters for recovering the oily fraction from the cereal germs and brans were:

For the test samples 1-4 listed in following Table 3:

TABLE 3

| | | |
|---|---|---|
| Extraction period: | >210 | minutes |
| Autoclave pressure: | >280 | bar |
| Pressure separator 1: | 65 | bar |
| Pressure separator 2: | 42 | bar |
| Autoclave temperature: | 65° | C. |
| Temperature separator 1: | 43° | C. |
| Temperature separator 2: | 26.8° | C. |

The oil contents of the brans were 2.2 to 3.6 % by weight.

For the test samples 5-7 listed in following Table 4:

TABLE 4
Single-stage extraction with single-stage extraction
separation

| | | |
|---|---|---|
| Autoclave pressure: | 260 | bar |
| Pressure separator: | 50 | bar |
| Autoclave temperature: | 45-50° | C. |
| Temperature separator: | 35° | C. |

Total CO₂ and CO₂ flow are dependent on the respective crude material used.

### EXAMPLE 2

Determination of the Tocopherols and Tocotrienols in the Germ- and Bran-Oils:

After saponification of the sample material and after extraction in n-hexane, the tocopherols and tocotrienols were separated by means of HPLC and detected by way of retention times with a fluorescence detector. The quantitative evaluation was effected by a comparison of the peak areas according to the external standard method. As the stationary phase, a HPLC column 250×4.6MMX1/4“VALCO; LiChrosorb Si60-5 was used, as mobile phase a mixture of n-hexane and dioxane (95:5) was used, and as comparative standards, tocopherol and tocotrienol from Calbiochem were used.

TABLE 5
The comparative analyses of the individual samples yielded the following results
(all values in mg/kg of test sample):

| Test Sample | | α-T¹⁾ | β-T²⁾ | γ-T³⁾ | δ-T⁴⁾ | α-T3⁵⁾ | β-T3⁶⁾ | γ-T3⁷⁾ | δ-T3⁸⁾ | Σ T⁹⁾ | Σ T3¹⁰⁾ | Σ E¹¹⁾ |
|---|---|---|---|---|---|---|---|---|---|---|---|---|
| 1. | Wheat germ | 3276 | 0 | 1048 | 0 | 0 | 124 | 0 | 0 | 4324 | 124 | 4448 |
| | oil from ungerminated | | | | | | | | | | | |
| | grain, extracted with | | | | | | | | | | | |
| | pressure separator 2 | | | | | | | | | | | |
| 2. | Wheat germ | 1339 | 283 | 14 | 14 | 833 | 1075 | 167 | 34 | 1650 | 2190 | 3759 |
| | oil, germinated with nutrient | | | | | | | | | | | |
| | solution for 96 | | | | | | | | | | | |
| | hours, extracted with | | | | | | | | | | | |
| | pressure separator 2 | | | | | | | | | | | |
| 3. | Wheat bran | 630 | 222 | 3 | 4 | 358 | 804 | 52 | 11 | 859 | 1224 | 2083 |
| | oil from ungerminated | | | | | | | | | | | |
| | grain, extracted with | | | | | | | | | | | |
| | pressure separator 1 | | | | | | | | | | | |
| 4. | Wheat bran | 91 | 0 | 6 | 0 | 799 | 424 | 71 | 20 | 97 | 1314 | 1411 |
| | oil, germinated with nutrient | | | | | | | | | | | |
| | solution for 96 hours, | | | | | | | | | | | |
| | extracted with | | | | | | | | | | | |
| | pressure separator 1 | | | | | | | | | | | |
| 5. | Barley bran | 264 | 0 | 149 | 12 | 893 | 140 | 275 | 33 | 425 | 1341 | 1766 |
| | oil, germinated for 24 | | | | | | | | | | | |
| | hours with water | | | | | | | | | | | |
| 6. | Barley bran | 143 | 10 | 43 | 8 | 1028 | 251 | 559 | 60 | 204 | 1898 | 2102 |
| | oil, germinated for 24 | | | | | | | | | | | |
| | hours with nutrient solution | | | | | | | | | | | |
| 7. | Barley bran | 88 | 0 | 59 | 6 | 320 | 78 | 156 | 22 | 153 | 576 | 729 |
| | oil, germinated for 96 | | | | | | | | | | | |
| | hours with nutrient | | | | | | | | | | | |
| | solution | | | | | | | | | | | |

Foot notes:
¹⁾alpha-tocopherol,
²⁾beta-tocopherol,
³⁾gamma-to-copherol,
⁴⁾delta-tocopherol;
⁵⁾alpha-tocotrienol,
⁶⁾beta-tocotrienol,
⁷⁾gamma-tocotrienol,
⁸⁾delta-tocotrienol;
⁹⁾Sum of tocopherols,
¹⁰⁾Sum of tocotrienols,
¹¹⁾Sum of vitamin E.

The results of the analyses show:
A decrease of the tocopherols in favor of an increase in the tocotrienols during germination, indicating a reduction of the cellular vitamin E activity with a simultaneous increase in the antioxidant demands during the germination process. On cell level, “germination” means an increased activity on enzymatic level. The plant cell requires a multiple of biologically highly valuable building materials, i.e. highly unsaturated fatty acids as well as phospholipids for the new formation of cell material, i.e. for growth. Due to the temperature and oxygen sensitivity of this highly valuable biological material, also the demand of antioxidant protective molecules increases. The present analysis results show that the plant cell meets this increased demand for protection by converting tocopherols to tocotrienols.
A stimulation of the endogenous tocotrienol synthesis in germinating seeds by supply of essential mineral micronutrients before and during germination:
   Whereas the plant cell—in contrast to the human cell—
is capable of meeting its (increased) demand of organic vital substances (vitamins, antioxidants, highly unsaturated fatty acids) autonomously by its own synthesis performance, it is—just like the human being—dependent on the exogenous supply of mineral substances (mineral substances, micronutrients). As the present tests show, the tocotrienol content of germinating seeds can be significantly increased by the supply of mineral micronutrients, as compared to conventional germination methods (i.e., germination with water).
A decrease of the tocopherol and tocotrienol contents with increasing duration of germination, demonstrated by the example of barley bran.

### EXAMPLE 3

Analysis of the Biological Quality of Natural Tocotrienol Mixtures:

Within the framework of an ex vivo-in vitro examination, the antioxidant capacity of tocotrienol-rich wheat bran oils is examined as compared to wheat germ oil, tocopherol acetate and D-alpha-tocopherol. In this examining method, human serum is subjected to the oxidative stress of a defined amount of para-benzoquinone. By adding defined amounts of the antioxidants to be tested (natural tocotrienol mixture of wheat bran oil, wheat germ oil, tocopherol acetat, D-alpha-tocopherol), the antioxidant load bearing capacity of the test sample is quantitated by calorimetric determination of the para-dihydroquinone that has been reduced from para-benzoquinone. After this test, the tocotrienol mixture of germinated wheat bran had an antixodidant capacity that was increased by the factor 500 as compared to tocopherol acetate, and an antioxidant capacity that was increased by the factor 1000 relative to D-alpha-tocopherol.

These tests were carried out as follows:

Human donor blood is recovered from the vein without any additives and centrifuged at 800-1000 g after having been left standing for 1 h in the refrigerator (approximately +4° C.-+7° C.). The separated serum fractions are removed and pooled. The serum can be stored at −22° C. for about 14 days or immediately be used for the required measurements.

To determine the antioxidant capacity of an antioxidant, the serum is radically loaded in stages. p-Benzoquinone was used for radical formation.

In a physiological environment (pH=slightly alkaline), this substance is converted into the relatively stable radical anion of the quinhydrone system. In doing so, one hydrogen atom (1 proton/1 electron) of p-benzoquinone is taken up.

The further reaction to the stable end product p-dihydroquinone takes place inversely proportionally, at pH 6.9-7.4 within minutes, and primarily linearly to the antioxidant content (reducing agent) in the reaction environment.

Thus, the conditions for the applicability of Lambert-Beer's law have been met for determining the end product (dihydro-quinone) as a typically coloured substance.

The comparatively stable radical anion (quinhydrone) is converted into the dihydroquinone in a second reduction step by taking up a further electrone (hydrogen from the available antioxidants). The reaction can calorimetrically be followed, since the transition to the completely reduced substance (dihydroquinone) involves a pronounced intensification of the colour.

The calorimetric determination of the amount of end product is effected at λ=500 nm. The extinction at this wave length then will be directly proportional to the amount of reaction end product, and also to the amount of reacted antioxidant for the conversion of the radical intermediate stage, respectively.

With a linear extinction increase-and a linearly growing consumption of the antioxidant-active substance, the reacted portions can be precisely calculated from the stoichiometric reaction conditions.

According to Michaelis/Kalkar and Pauling, respectively (Holleman-Wiberg; Lehrbuch der Anorganischen Chemie, de Gruyter-Verlag (1995)), the reaction intensity up to the dihydroquinone will depend on the ratio of the concentrations of oxidised to reduced. I.e., the higher the reduction (antioxidant) supply, the more the reaction will be inhibited, the content of the dihydroquinone forming will decrease. Thus, this reaction is excellently suited to be used in redox systems for finely dosed quantitative determinations for substances that are reducingly active.

Procedure:

In three parallel measurement series, serum samples (500 ml each) of the pool with p-benzoquinone are loaded in three stages (10, 20 and 30 μg). After a reaction time of 30, 60, 90 and 180 seconds, the respective extinction values at λ=500 nm are determined. In the calculated reaction quantities, the mean of the 60 second value corresponds to the defined titer (=one third reaction). This value precisely results from the graphic illustration of the mean values as extinction curve. All further tests with antioxidant addition are comparatively related to this defined calibration value of the serum pool without antioxidant addition (i.e., with an increasing supply of antioxidants in the serum, the extinction must drop for dihydroquinone).

Three parallel measurement series of serum samples were carried out under addition of 500 μg/ml of preparation according to the invention to determine the antioxidant capacity of the tocotrienol-rich wheat bran oils according to the invention. There resulted a decrease in the extinction value (λ=500 nm/60 sec) by a mean of 0.017 units as compared to the non-treated serum.

Referring to the curve of the extinction values of the comparative serum without additions and the stoichiometric reaction conditions applied (serum determination/concentration of p-benzoquinone/amount per ml of serum dilution), the conversion factor extinction to radical inhibition will result from this calibration system.

With the employed 1/50 dilution of the comparative serum samples and the one-third reaction (extinction comparison) of the number of molecules used (to be calculated from the p-benzoquinone concentration per volume unit according to Loschmidt's number), there results the conversion factor to quinhydron radicals. According to this, 0.017 extinction units correspond to 0.017·10·28.08=4.914 μg radicals per ml/sec.

In the formulation:
3 mg of preparation according to the invention in 6 ml of serum, 0.5 ml thereof diluted 1:10 isotonic give 250 μg in 5 ml. The extinction measurements were carried out in 1.5 ml thereof with additions of 10 or 20 and 30 μg of p-benzoquinone, respectively, which corresponds to an amount of 75 μg of preparation according to the invention.
75 μg of inventive preparation=4.914 μg of radicals per ml/sec
1 mg of inventive preparation=65.52 μg of radicals as well as
1 g of inventive preparation=65.52 mg of radicals per ml and sec.
as detoxicating performance of the preparation according to the invention.

This activity can be calculated for to the oxygen stages as desired, by means of Loschmidt's number: 1 g of inventive oil=the detoxication of 23.28-10²⁰ OH.-radicals (atomic weight=17) or
12.37·10²⁰
O₂.-radicals (atomic weight=32)

A further criterion for the evaluation of the antioxidant (protective) action is (the influence on) redox buffering. Physiologically active substances having an antioxidant effect stabilise or increase the oxidative load bearing capacity (loading) of biological oxido-reductive systems (serum, e.g.) despite an increasing radical load.

To determine the activity of the preparation according to the invention for stabilising the oxidative load bearing capacity of biological systems, again three test series are examined, and the mean values are determined therefrom:

1. Human Whole Serum (Vital) Without Preparation According to the Invention (Blank Serum)

Serum samples of 0.5 ml volume each are loaded with 10, 20 or 30 μg, respectively, of p-benzoquinone. After incubating for 30 min (20° C.), the redox potentials (mV) are measured, compared with the blank value potential (without p-benzoquinone) and graphically illustrated as buffer curve. The buffer function results as a linear function between the potential points for 10, 20 and 30 μg of load, respectively, by p-benzoquinone.

2. Human Whole Serum (Vital) With 500 μg of Inventive Preparation Per ml

The serum samples of 0.5 ml each were also loaded with 10 or 20 and 30 μg respectively, of p-benzoquinone. After incubating for 30 min, the redox potentials were measured and graphically illustrated in a comparison with the potential value without p-benzoquinone. Here, too, the buffer function results as a linear equation of the connection of the points for 10, 20 and 30 μg of p-benzoquinone.

Result:

For serum with an addition of inventive preparation, there results a rise ratio in the buffer curve of +0.35 mV. Without inventive preparation, the rise ratio of the linear buffer function is only −0.35 mV.

Thus, the product according to the invention is a highly effective antioxidant also under physiological conditions (also in vivo).

For a more encompassing evaluation of the product quality, the comparison with other antioxidants is required. For this purpose, analogously three analysis series were carried out as described above for the products:
wheat germ oil (virgin, 100% purest pharmacy quality), D,L-alpha-tocopherol acetate (synthetic, 50%)—(visvitalis (AT)),
alpha-tocopherol (natural, 50%)—(vis-vitalis (AT)).

The results are summarised in the following Table 6.

TABLE 6

| | Wheat | | | |
|---|---|---|---|---|
| | germ oil | | | |
| | according to | Wheat | Tocopherol | D-alpha- |
| | the invention | germ oil | acetate | Tocopherol |
| Antiox-Titer | 0.19 | 0.14 | 0.125 | 0.06 |
| Radicals | 5.335 | 3.92 | 3.51 | 1.6848 |
| μg/ml · sec | | | | |
| Tocotrienols at | 850 μg | 1.4 mg | | 250 mg |
| 500 mg | | | | |
| substance/ml | | | | |
| 1 mg of tocotrienol | 6.276 | 2.8 | 0.014 | 0.00674 |
| effect in μg | | | | |
| radicals/ml · sec. | | | | |
| Factor of efficacy | 1 | 0.446 | 0.0022 | 0.001 |
| | | (1/2.24) | (1/450) | (1/930) |

From all the tests, the following can be stated:

The preparation according to the invention is highly effective as an antioxidant protective product and, likewise, unfolds a high protective function against super-oxidation also under physiological conditions, by increasing the redox buffering (loading) of biological redox systems.

Conclusion:

In contrast to tocopherols, tocotrienols have a lower vitamin E activity, yet they have a markedly increased antioxidant performance. The antioxidant capacity of lipophilic antioxidants is an important quality parameter in their nutritional-medical use for immune, heart/circulatory, muscle/joint, liver, skin and nerve diseases. Germs and brans from cereal and leguminose seeds have lower tocopherol, yet higher tocotrienol contents as compared to non-germinated seeds. In comparison to distilled water, a plant's synthesis of tocotrienols can be markedly stimulated during the germination process by the inventive addition of essential mineral micronutrients.

1-15. (canceled)

16. A method of producing a tocotrienol-enriched preparation, comprising:
incubating plant seeds with an electrolyte nutrient solution to form tocotrienol-enriched plant embryos; and
extracting a tocotrienol-containing preparation from the plant embryos.

17. The method of claim 16, wherein the tocotrienol-containing preparation is extracted from bran and germ of the plant embryos.

18. The method of claim 16, wherein the plant seeds are walnut, wheat, sunflower, palm, rye, barley, oat, amaranth, quinoa, and/or rice seeds.

19. The method of claim 16, wherein the plant embryos are dried prior to extraction.

20. The method of claim 16, wherein the plant embryos are ground prior to extraction.

21. The method of claim 16, further defined as extracting an oily extract from the plant embryos.

22. The method of claim 16, wherein the extraction is effected with supercritical CO₂ and/or hexane.

23. The method of claim 16, wherein incubation is at a temperature of from 1I0C to 40° C.

24. The method of claim 23, wherein incubation is at a temperature of from 15C to 30° C.

25. The method of claim 24, wherein incubation is at a temperature of from 19° C. to 21° C.

26. The method of claim 16, wherein the nutrient solution contains vanadium, selenate, molybdate, cobalt, chromium(III), manganese, strontium, lithium, copper iron(III), zinc, gluconate, citrate, and/or lactate ions in an amount of from 0.I to 1000 mg/l.

27. The method of claim 26, wherein the nutrient solution contains vanadium, selenate, molybdate, cobalt, chromium(III), manganese, strontium, lithium, copper iron(III), zinc, gluconate, citrate, and/or lactate ions in an amount of from 1 to 500 mg/I.

28. The method of claim 27, wherein the nutrient solution contains vanadium, selenate, molybdate, cobalt, chromium(III), manganese, strontium, lithium, copper iron(III), zinc, gluconate, citrate, and/or lactate ions in an amount of from 3 to 100 mg/l.

29. The method of claim 16, wherein the extraction is carried out with an autoclave and a pressure separator.

30. The method of claim 29, wherein the autoclave pressure is 100 bar or more, the separator pressure is 20 bar or more, the autoclave temperature is 30° C. or more, and the separator temperature is 20° C. or more.

31. The method of claim 30, wherein the autoclave pressure is 200 bar or more.

32. The method of claim 31, wherein the autoclave pressure is 250 bar or more.

33. The method of claim 30, wherein the separator pressure is 30 bar or more.

34. The method of claim 33, wherein the separator pressure is 45 bar or more.

35. The method of claim 30, wherein the autoclave temperature is 40° C. or more.

36. The method of claim 35, wherein the autoclave temperature is 50° C. or more.

37. The method of claim 30, wherein the separator temperature is 30° C. or more.

38. The method of claim 37, wherein the separator temperature is 40° C. or more.

39. The method of claim 16, wherein the nutrient solution is changed at least once during incubating.

40. The method of claim 39, wherein the nutrient solution is changed at least twice during incubating.

41. The method of claim 40, wherein the nutrient solution is changed at least three times during incubating.

42. A tocotrienol-enriched wheat embryo preparation from the plant embryo and/or from the bran of wheat embryos, comprising a tocotrienol content of at least 500 mg/g dry material.

43. The tocotrienol-enriched wheat embryo preparation of claim 42, further comprising at least 1000 mg/kg dry material.

44. The tocotrienol-enriched wheat embryo preparation of claim 43, further comprising at least 2000 mg/kg dry material.

45. The tocotrienol-enriched wheat embryo preparation of claim 42, further comprising a pharmaceutically active substance, a pharmaceutical adjuvant, a food-technological product and/or a food-technological additive.

46. A tocotrienol enriched barley embryo preparation from the plant embryo and/or from the bran of barley embryos, comprising a tocotrienol content of at least 1500 mg/kg dry material.

47. The tocotrienol-enriched barley embryo preparation of claim 46, further comprising a gamma-tocotrienol content of at least 500 mg/kg dry material.

48. The tocotrienol-enriched barley embryo preparation of claim 46, further comprising a beta-tocotrienol content of at least 200 mg/kg dry material.

49. The tocotrienol-enriched barley embryo preparation of claim 46, further comprising a pharmaceutically active substance, a pharmaceutical adjuvant, a food-technological product and/or a food-technological additive.
